(19) United States
(12) Patent Application Publication
(10) Pub. No.: US 2012/0283283 A1
(43) Pub. Date: Nov. 8, 2012

(54) METHODS FOR DETECTING ENHANCED RISK OF OPIOID-INDUCED HYPOXIA IN A PATIENT

(76) Inventors: Warren C. Stern, Plymouth, MA (US); Patricia T. Richards, West New York, NJ (US)

(21) Appl. No.: 13/464,025

(22) Filed: May 4, 2012

Related U.S. Application Data
(60) Provisional application No. 61/483,024, filed on May 5, 2011.

(51) Int. Cl. G01N 33/50; A61P 25/00; A61K 31/485

(52) U.S. Cl. 514/282; 436/68

(57) ABSTRACT

Methods for detecting enhanced risk of opioid-induced respiratory dysfunction in patients with normal levels of oxygen saturation. The method may comprise: (1) assaying blood of the patient for a normal level of oxygen saturation; (2) measuring the patient's respiration rate at rest for a normal rate; and (3) correlating the normal level of oxygen saturation and the normal rate of respiration with enhanced risk of opioid-induced respiratory dysfunction if: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid. The method may further comprise administering opioids to a patient after detecting whether there is an enhanced risk of opioid-induced respiratory dysfunction.

### CROSS-REFERENCE TO RELATED APPLICATIONS

This application claims priority to U.S. provisional application Ser. No. 61/483,024, filed May 5, 2011, the entirety of which is incorporated herein by reference.

### FIELD OF THE INVENTION

This invention relates to methods for detecting enhanced risk of opioid-induced respiratory dysfunction in patients having normal respiratory function and who are to be treated with opioids for pain. This invention further relates to methods for treating pain and for administering opioids to patients who have been identified as having an enhanced risk of opioid-induced respiratory dysfunction.

### BACKGROUND OF THE INVENTION

Patients who experience significant pain as the result of, for example, a serious traumatic injury, a surgical procedure, or chronic illness (e.g., cancer), often require analgesic relief through strong prescription medication. Opioid drugs are a class of pain-relieving prescription medications frequently used in the treatment of acute and chronic, moderate to severe, pain.

Opioid analgesics are widely recognized for interacting with the respiratory system, in particular for increasing the risk of respiratory depression. See Dahan et al., Anesthesiology 2010,112, 226-38; Cashman et al., Br. J. Anaesth. 2004, 93, 212-23; Pattinson, Br. J. Anaesth. 2008, 100, 747-58. Opioids can adversely impact respiratory function even in healthy patients, potentially resulting in dose-dependent increases in the rate (proportion of patients) of blood oxygen desaturation (SpO₂<90%), low respiratory rates (<10 breaths/min) and need for rescue by an opioid receptor blocker. See Wheeler et al., J. Pain 2002, 3, 159-180. Adverse outcomes from oxygen desaturation and respiratory depression may include loss of consciousness, respiratory arrest, myocardial infarction, seizures and death. See Dahan at 228; Wheeler at 161.

Such outcomes can be prevented through careful monitoring of patients' respiratory functions during treatment with an opioid analgesic. See Dahan at 228. Patients with opioid-induced respiratory dysfunction can sometimes reestablish normal respiratory rates without intervention, while others require oxygen supplementation or even mechanical ventilation for several hours before returning to baseline. See Wheeler at 161. Opioid-induced respiratory dysfunction may also be reversed by the use of blocking agents such as naloxone or naltrexone. See Dahan at 228.

Under most circumstances such events are infrequent, but the risk of opioid-induced respiratory dysfunction has been known to vary depending on the opioid and the dosage. In one study, patients self-administered morphine (1 mg bolus doses with a 5-10 min lockout period) for postoperative pain via patient-controlled analgesia (PCA), whereby about 2% of subjects experienced respiratory dysfunction. See Dahan at 228. In another study, 6% of subjects with normal baseline oxygen saturation levels had recorded oxygen saturation levels of less than 90% after being treated intravenously with 2 mg of hydromorphone in a 2-3 minute period for postoperative pain. See Chang, J. Opioid Manag., 2009, 5, 75-80.

In order to ensure safe administration of opioids, continual monitoring of a patient's respiratory status is often advised if the patient is deemed to be at increased risk of opioid-induced respiratory dysfunction based on pre-existing respiratory illnesses, impaired consciousness, or a history of opioid-induced respiratory impairment, for example. See Wheeler at 161. However, routine continuous monitoring of respiratory function has limitations on financial and equipment resources, as well as on medical staff time in the event that frequent observation of the patient is needed, thus rendering continual monitoring for all opioid treated patients nearly impossible. Further, continual monitoring may be difficult if an opioid analgesic is administered at home, as is often the case after outpatient surgery or discharge from the hospital following a major surgical procedure. Therefore, a method to identify those patients who are at enhanced risk of opioid-induced respiratory dysfunction would benefit both inpatients and outpatients who are in need of opioid therapy for the treatment of pain. In the case of outpatients, the ability to identify high-risk patients would better prepare medical staff when treating patients with opioids and minimize the occurrence of moderate-severe respiratory related adverse events by allowing the medical staff to focus on patients who are at most risk and to implement counter measures should significant respiratory depression occur. In respect to outpatients who are not subject to direct monitoring by medical staff or hospital equipment, the dose of the opioid medication prescribed could be lower than that which would otherwise have been given to optimally manage pain in order to reduce the risk of a major respiratory impairment.

### SUMMARY OF THE INVENTION

The present invention relates to methods of detecting enhanced risk of opioid-induced respiratory dysfunction in a patient to be treated for pain. These methods address the unmet need to identify patients with normal baseline respiratory functions who may be at an enhanced risk of opioid-induced respiratory dysfunction prior to, or during, treatment for pain. By identifying such patients, adverse events associated with opioid-induced respiratory dysfunction, including loss of consciousness, respiratory arrest, seizures and death, can be reduced or prevented.

An aspect of the invention is therefore directed to a method for detecting enhanced risk of opioid-induced respiratory dysfunction in a patient to be treated for pain, comprising:

(1) assaying blood of the patient for a normal level of oxygen saturation, wherein the normal level of oxygen saturation is about 90% or greater;

(2) measuring the patient's respiration for a normal rate, wherein the normal rate of respiration at rest is greater than about 10 and less than about 20 breaths per minute; and

(3) correlating the normal level of oxygen saturation and the normal rate of respiration with enhanced risk of opioid-induced respiratory dysfunction if: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid.

In some embodiments, if the patient is at enhanced risk of opioid-induced respiratory dysfunction, the treatment for pain may be administered at a lower dose or a lower-than-normal dose; or the treatment for pain may be initially administered at a lower dose or a lower-than-normal dose, and than titrated upwards at a slower, or slower than normal, titration rate; or the patient may be administered oxygen; or the patient may cease the pain treatment; or the patient may be monitored more closely.

Another aspect of the invention is directed to a method for administering at least one opioid to a patient comprising:

(1) assaying blood of the patient for a normal level of oxygen saturation, wherein the normal level of oxygen saturation is about 90% or greater;

(2) measuring the patient's respiration for a normal rate, wherein the normal rate of respiration at rest is greater than about 10 and less than about 20 breaths per minute; and

(3) correlating the normal level of oxygen saturation and the normal rate of respiration with enhanced risk of opioid-induced respiratory dysfunction if: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid;

(4) administering to the patient a formulation comprising at least one opioid; and

(5) monitoring the patient's oxygen saturation level if the patient is determined to be at enhanced risk of opioid-induced respiratory dysfunction, and administering supplemental oxygen if the oxygen saturation level is less than about 90%.

In certain embodiments, an enhanced risk of opioid-induced respiratory dysfunction in a patient with normal oxygen saturation and normal rate of respiration may be correlated with two or more of the following factors: (a) the patient is at an altitude of about 1000 feet above sea level or greater; and/or (b) the patient's oxygen saturation level is normal but no greater than about 95%; and/or (c) the patient received prior dosing with intravenous opioid and is converted to dosing with an oral opioid.

In some embodiments of the present invention, a patient's blood may be assayed for a level of blood oxygen saturation (sometimes referred herein as a “baseline” level of blood oxygen saturation), wherein the range of the oxygen saturation level is greater than about 90%, greater than about 91%, greater than about 92%, greater than about 93%, greater than about 94%, greater than about 95% or greater than about 96%.

In some embodiments of the present invention, a patient's respiratory rate may be measured for a normal rate, wherein the range of respiratory rates may be selected from the group consisting of greater than about 11 and less than about 20 breaths per minute, greater than about 12 and less than about 20 breaths per minute, greater than about 13 and less than about 20 breaths per minute and greater than about 14 and less than about 20 breaths per minute.

In some embodiments of the present invention, methods may comprise correlating a normal level of oxygen saturation and a normal rate of respiration with an enhanced risk of opioid-induced respiratory dysfunction if the patient is at an elevated altitude above sea level, wherein the altitude is about 2000 feet above sea level or greater, about 3000 feet above sea level or greater or about 4000 feet above sea level or greater.

In certain embodiments, methods may comprise correlating oxygen saturation, which is a baseline oxygen saturation, and a normal rate of respiration with an enhanced risk of opioid-induced respiratory dysfunction if the patient's oxygen saturation level (SpO₂) is not greater than about 95%, about 94%, about 93%, about 92%, about 91% or about 90%.

In some embodiments, methods may comprise correlating a normal level of oxygen saturation and a normal rate of respiration with an enhanced risk of opioid-induced respiratory dysfunction if the patient received prior dosing with an intravenous opioid and is converted to dosing with an oral opioid. In other embodiments, the patient is converted to dosing with an oral opioid from a prior intravenous opioid dosing, wherein the prior intravenous opioid dosing was administered for a duration of at least about 48 hours, at least about 24 hours, at least about 20 hours, at least about 16 hours, at least about 12 hours, at least about 8 hours, at least about 6 hours, at least about 4 hours, at least about about 2 hours or at least about 1 hour.

In some embodiments, the oral opioid is administered within about 15 minutes following the end of prior intravenous opioid dosing. In some embodiments, the oral opioid is administered within a time range of prior intravenous opioid dosing selected from the group consisting of about 15 minutes to about 30 minutes, about 30 minutes to about 1 hour, about 1 hour to about 2 hours, about 2 hours to about 4 hours, about 4 hours to about 8 hours, about 8 hours to about 12 hours, about 12 hours to about 16 hours, about 16 hours to about 24 hours and about 24 hours to about 48 hours. In another embodiment, enhanced risk of opioid-induced respiratory dysfunction may occur if a patient is converted to dosing with an oral opioid from a prior intravenous opioid dosing within about 12 hours of prior intravenous opioid dosing, within about 8 hours of prior intravenous opioid dosing, within about 4 hours of prior intravenous opioid dosing, within about 2 hours of prior intravenous opioid dosing, within about 1 hour of prior intravenous opioid dosing or within about 30 minutes of prior intravenous opioid dosing.

In certain embodiments, the prior intravenous opioid dosing comprises one ore more opioids selected from the group consisting of codeine, thebaine, hydromorphone, hydrocodone, oxycodone, oxymorphone, diacetylmorphine (heroin), nicomorphine, dipropanoylmorphine, benzylmorphine, ethylmorphine, buprenorphine and morphine glucuronides (including the 3- and 6-glucuronide), alfentanil, fentanyl, remifentanil, sufentanil, trefentanil, pethidine, methadone, tramadol, dextropropoxyphene, tapentadol, a pharmaceutically acceptable salt thereof, and a combination thereof. In particular embodiments, the prior intravenous opioid dosing is morphine, or a pharmaceutically acceptable salt thereof, or oxycodone, or a pharmaceutical salt thereof.

In certain embodiments, the prior intravenous opioid dosing comprises a combination of at least two opioids. In certain embodiments, the prior intravenous opioid dosing is a combination of two opioids. In certain embodiments, the prior intravenous opioid dosing is a combination comprising morphine, or a pharmaceutically acceptable salt thereof, and oxycodone, or a pharmaceutically acceptable salt thereof. In some embodiments, the prior intravenous opioid dosing comprises morphine, or a pharmaceutically acceptable salt thereof, and oxycodone, or a pharmaceutically acceptable salt thereof in a ratio of about 3:1 to 1:3 (weight:weight). In certain embodiments, the prior intravenous opioid dosing comprises morphine, or a pharmaceutically acceptable salt thereof, and oxycodone, or a pharmaceutically acceptable salt thereof in a ratio of about 3:2 (weight:weight).

In some embodiments of the present invention, the assay for determining the level of oxygen saturation in a patient's blood comprises performing pulse oximetry or an arterial blood gas test. In certain embodiments, the assay for determining the level of oxygen saturation in a patient comprises performing an arterial blood gas test. In certain embodiments, the assay for determining the level of oxygen saturation in a patient comprises performing pulse oximetry.

In certain embodiments, the at least one opioid in the formulation is selected from the group consisting of codeine, thebaine, hydromorphone, hydrocodone, oxycodone, oxymorphone, diacetylmorphine (heroin), nicomorphine, dipropanoylmorphine, benzylmorphine, ethylmorphine, buprenorphine and morphine glucuronides (including the 3- and 6-glucuronide), alfentanil, fentanyl, remifentanil, sufentanil, trefentanil, pethidine, methadone, tramadol, dextropropoxyphene, a pharmaceutically acceptable salt thereof, and a combination thereof. In particular embodiments, the opioid is morphine, or a pharmaceutically acceptable salt thereof, or oxycodone, or a pharmaceutical salt thereof.

In some embodiments of the present invention, the formulation comprises a combination of at least two opioids. In certain embodiments, the formulation comprises a combination of two opioids. In some embodiments, the formulation comprising two opioids may be administered intravenously. In other embodiments, the formulation comprising two opioids may be administered orally. In certain embodiments, the formulation comprises morphine, or a pharmaceutically acceptable salt thereof, and oxycodone, or a pharmaceutically acceptable salt thereof. In some embodiments, the formulation comprises morphine, or a pharmaceutically acceptable salt thereof, and oxycodone, or a pharmaceutically acceptable salt thereof in a ratio of about 3:1 to 1:3 (weight:weight). In certain embodiments, the formulation comprises morphine, or a pharmaceutically acceptable salt thereof, and oxycodone, or a pharmaceutically acceptable salt thereof in a ratio of about 3:2 (weight:weight). In some embodiments, the morphine-oxycodone combination may be co-administered in separate formulations.

In certain embodiments, the pharmaceutically acceptable salt may be a hydrochloride, hydrobromide, hydroiodide, sulfate, bisulfate, nitrate, citrate, tartrate, bitartrate, phosphate, malate, maleate, napsylate, fumarate, succinate, acetate, terephthalate, pamoate or pectinate.

In particular embodiments, the formulation comprises morphine sulfate and oxycodone hydrochloride.

In the present invention, the formulation comprising at least one opioid may be administered orally or parenterally. In certain embodiments, the parenteral administration is intravenous (IV) administration.

In some embodiments, the formulation administered orally may be in an immediate release, sustained release or controlled release dosage form.

In some embodiments, the formulation comprising morphine and oxycodone may be in an immediate release dosage form, sustained release dosage form, or controlled release dosage form. In particular embodiments, the formulation comprising morphine and oxycodone may be in an immediate release dosage form.

Another aspect of this invention is a method of treating pain in a subject with an enhanced risk of opioid-induced respiratory dysfunction, which comprises:

(1) identifying a patient with an enhanced risk of opioid-induced respiratory dysfunction by: (a) assaying blood of the patient for a normal level of oxygen saturation, wherein the normal level of oxygen saturation is about 90% or greater; and (b) measuring the patient's respiration for a normal rate, wherein the normal rate of respiration is greater than about 10 and less than about breaths per minute; and (c) correlating the normal level of oxygen saturation and the normal rate of respiration with enhanced risk of opioid-induced respiratory dysfunction if: (i) the patient is at an altitude of about 1000 feet above sea level or greater; or (ii) the patient's oxygen saturation level is normal but no greater than about 95%; or(iii) the patient received prior dosing with intravenous opioid and is converted to dosing with an oral opioid;

(2) administering a formulation of two or more opioids concurrently to the patient if the patient is determined to be at enhanced risk of opioid-induced respiratory dysfunction.

The method may further comprise monitoring the patient's oxygen saturation level if the patient is determined to be at enhanced risk of opioid-induced respiratory dysfunction, and administering supplemental oxygen if the oxygen saturation level is less than about 90%.

A further aspect of the invention is a formulation comprising at least one opioid for use in treating pain in a patient at enhanced risk of opioid-induced respiratory dysfunction, wherein the patient has a normal level of oxygen saturation of about 90% or greater, and a normal rate of respiration at rest which is greater than about 10 and less than about 20 breaths per minute, and either: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid; and wherein the formulation comprising as least one opioid is for use in combination with supplemental oxygen if the blood oxygen saturation level is less than about 90% following opioid administration.

Yet, another aspect of the invention is a formulation comprising at least one opioid for use in combination with supplemental oxygen for treating pain in a patient at enhanced risk of opioid-induced respiratory dysfunction, wherein prior to administration of the opioid the patient has a normal level of oxygen saturation of about 90% or greater, and a normal rate of respiration at rest which is greater than about 10 and less than about 20 breaths per minute, and either: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid; and wherein following administration of the opioid the blood oxygen saturation level drops to less than about 90%.

In certain embodiments, the formulation comprises two opioids that are co-administered to a patient. In some embodiments, the formulation comprises morphine, or a pharmaceutically acceptable salt thereof, and oxycodone, or a pharmaceutically acceptable salt thereof. In other embodiments, the formulation comprises morphine, or a pharmaceutically acceptable salt thereof, and oxycodone, or a pharmaceutically acceptable salt thereof in a ratio of 3:2 by weight.

In alternative embodiments, the method comprises administering two or more opioids concurrently in separate formulations.

These and other objects, features and advantages of the present invention will become apparent after a review of the following detailed description.

### BRIEF DESCRIPTION OF THE DRAWINGS

FIG. 1 shows patients treated with an opioid at high altitude sites (>about 4000 feet above sea level) having SpO₂<90%.

FIG. 2 shows patients treated with an opioid having SpO₂<90% that were not located at a high altitude site.

FIG. 3 shows combined subjects treated with an opioid experiencing SpO₂<90% as a function of baseline SpO₂ saturation level.

### DETAILED DESCRIPTION

The present invention provides for safer administration of opioid analgesics in patients with normal (baseline) levels of oxygen saturation and normal respiration rates by detecting whether the patient has an enhanced risk of opioid-induced respiratory dysfunction. The invention was developed based on a discovery that patients associated with certain factors will have a clinically enhanced risk of experiencing opioid-induced respiratory dysfunction. By identifying these patients with enhanced risk of opioid-induced respiratory dysfunction prior to or during treatment, adverse events may be prevented.

As disclosed herein, it is an aspect of this invention that certain measurable factors may be correlated with an enhanced risk of opioid-induced respiratory dysfunction in patients with normal levels of oxygen saturation and normal respiration rates. Enhanced risk of opioid-induced-respiratory dysfunction may be correlated with factors including, but not limited to, altitude at which the patient resides and is treated with an opioid, (baseline) oxygen saturation levels of the patient that are between about 90 and about 94%, or no greater than about 95%, and/or intravenous administration of an opioid to patient, prior to administration of an oral opioid dosage.

As used herein in the context of a number, or a range of numbers, “about” will be understood to embrace somewhat larger or smaller values than the indicated value to account for, as examples, experimental errors inherent in the measurement and variability between different methodologies for measuring the value, as will be apparent to one skilled in the art.

As used herein, “administration concurrently” or “co-administration” refers to the administration of a single composition containing two or more opioids or pharmaceutically acceptable salts thereof, or to the administration of each opioid agonist as a separate composition within a short enough period of time such that the effective result is equivalent to that obtained when both such opioid agonists are administered as a single composition.

As used herein, “enhanced risk” of an opioid-induced respiratory dysfunction, as associated with the presence of a factor, is defined as a clinically greater likelihood that a patient will experience opioid-induced respiratory dysfunction than if the factor was not present. In certain embodiments, the clinically greater likelihood of experiencing opioid-induced respiratory dysfunction may be at least about 25% greater likelihood, at least about 50% greater likelihood, at least about 100% greater likelihood, at least about 200% greater likelihood, at least about 300% greater likelihood, at least about 400% greater likelihood, at least about 500% greater likelihood, at least about 600% greater likelihood, at least about 700% greater likelihood, at least about 800% greater likelihood, at least about 900% greater likelihood or at least about 1000% greater likelihood.

As used herein, “morphine equivalent dose” refers to a calculation of the amount of morphine that produces the same analgesic effects as a particular amount of another opioid for given route(s) of dose administration. For example, the oral morphine equivalent dose of 1 mg of oral oxycodone is 1.5 mg of oral morphine; in other words, 1 mg of oxycodone administered orally will provide the same analgesic effect as 1.5 mg of morphine administered orally.

As used herein, “morphine-oxycodone combination” refers to a combination of morphine, or a pharmaceutically acceptable salt thereof, and oxycodone, or a pharmaceutically acceptable salt thereof.

As used herein, “normal levels of oxygen saturation” refers to oxygen saturation levels of at least about 90% (SpO₂≧90%).

As used herein, “normal respiration rates” at rest refers to respiratory rates of about 10 to about 20 breaths per minute at rest.

As used herein, “opioid-induced respiratory dysfunction,” refers to adverse effects on respiration induced by administration one or more opioids, in particular respiratory desaturation or abnormal respiration rate.

As used herein, “oxygen desaturation” refers to oxygen saturation levels that are below normal to a clinically significant extent, in particular, oxygen saturation levels that are below about 90% (SpO₂<90%).

As used herein, “pharmaceutically acceptable salt” refers to a salt that is toxicologically safe for human and animal administration.

Examples of opioids include, but are not limited to, natural opiates such as morphine, codeine, and thebaine; semi-synthetic opioids such as hydromorphone, hydrocodone, oxycodone, oxymorphone, diacetylmorphine (heroin), nicomorphine, dipropanoylmorphine, benzylmorphine, ethylmorphine, buprenorphine and morphine glucuronides (including the 3- and 6-glucuronide); and synthetic opioids such as alfentanil, fentanyl, remifentanil, sufentanil, trefentanil, pethidine, methadone, tramadol and dextropropoxyphene.

The present invention relates to methods of detecting enhanced risk of opioid-induced respiratory dysfunction in a patient to be treated for pain. Opioid-induced respiratory dysfunction can result in oxygen desaturation and abnormal respiration rates; it can sometimes also lead to adverse events such as loss of consciousness, respiratory arrest, myocardial infarction, seizures and death. In patients to be treated for pain with normal (baseline) levels of oxygen saturation and normal respiration rates, there are certain measurable factors which may contribute to an enhanced risk of opioid-induced respiratory dysfunction including without limitation, elevated altitude, a normal oxygen saturation level that is below about 95% (SpO₂<95%), and previous administration of intravenous opioids prior to administration of oral opioids. The present invention makes it possible to correlate these factors with an enhanced risk of opioid-induced respiratory dysfunction.

An aspect of the invention is directed to a method for detecting enhanced risk of opioid-induced respiratory dysfunction in a patient to be treated for pain, comprising:

(1) assaying blood of the patient for a normal level of oxygen saturation, wherein the normal level of oxygen saturation is about 90% or greater;

(2) measuring the patient's respiration for a normal rate, wherein the normal rate of respiration at rest is greater than about 10 and less than about 20 breaths per minute; and

(3) correlating the normal level of oxygen saturation and the normal rate of respiration with enhanced risk of opioid-induced respiratory dysfunction if: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid.

In some embodiments, if the patient is at enhanced risk of opioid-induced respiratory dysfunction, the treatment for pain may be administered at a lower dose or a lower-than-normal dose; or the treatment for pain may be initially administered at a lower dose or a lower-than-normal dose, and than titrated upwards at a slower, or slower than normal, titration rate; or the patient may be administered oxygen; or the patient may cease the pain treatment; or the patient may be monitored more closely.

Another aspect of the invention is directed to a method for administering at least one opioid to a patient comprising:

(1) assaying blood of the patient for a normal level of oxygen saturation, wherein the normal level of oxygen saturation is about 90% or greater;

(2) measuring the patient's respiration for a normal rate, wherein the normal rate of respiration at rest is greater than about 10 and less than about 20 breaths per minute; and

(3) correlating the normal level of oxygen saturation and the normal rate of respiration with enhanced risk of opioid-induced respiratory dysfunction if: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid;

(4) administering to the patient a formulation comprising at least one opioid; and

(5) monitoring the patient's oxygen saturation level if the patient is determined to be at enhanced risk of opioid-induced respiratory dysfunction, and administering supplemental oxygen if the oxygen saturation level is less than about 90%.

In certain embodiments, an enhanced risk of opioid-induced respiratory dysfunction in a patient with normal oxygen saturation and normal rate of respiration may be correlated with two or more of the following factors: (a) the patient is at an altitude of about 1000 feet above sea level or greater; and/or (b) the patient's oxygen saturation level is normal but no greater than about 95%; and/or (c) the patient received prior dosing with intravenous opioid.

In addition, administering two or more opioids to a patient concurrently may reduce the clinical likelihood of opioid-induced respiratory dysfunction, when compared to administration of a single opioid with the same or lower morphine equivalent dose. For example, when patients are co-administered a combination of morphine and oxycodone, the morphine equivalent dose required to induce oxygen desaturation was higher than either oxycodone or morphine alone. Additionally, a lower percentage of patients suffered from oxygen desaturation when administered a morphine-oxycodone combination than those patients administered morphine or oxycodone alone, wherein all patients received approximately the same morphine equivalent dose.

Yet another aspect of this invention is a method of treating pain in a subject with an enhanced risk of opioid-induced respiratory dysfunction, which comprises:

(1) identifying a patient with an enhanced risk of opioid-induced respiratory dysfunction by: (a) assaying blood of the patient for a normal level of oxygen saturation, wherein the normal level of oxygen saturation is about 90% or greater; and (b) measuring the patient's respiration for a normal rate, wherein the normal rate of respiration is greater than about 10 breaths per minute; and (c) correlating the normal level of oxygen saturation and the normal rate of respiration with enhanced risk of opioid-induced respiratory dysfunction if: (i) the patient is at an altitude of about 1000 feet above sea level or greater; or (ii) the patient's oxygen saturation level is normal but no greater than about 95%; or (iii) the patient received prior dosing with intravenous opioid and is converted to dosing with an oral opioid;

(2) administering a formulation of two or more opioids concurrently to the patient if the patient is determined to be at enhanced risk of opioid-induced respiratory dysfunction.

The method may further comprise monitoring the patient's oxygen saturation level if the patient is determined to be at enhanced risk of opioid-induced respiratory dysfunction, and administering supplemental oxygen if the oxygen saturation level is less than about 90%.

Normal Respiration

In some embodiments of the present invention, a patient's blood may be assayed for a normal level of oxygen saturation, wherein the range of the oxygen saturation level is greater than about 90%, or greater than about 91%, greater than about 92%, greater than about 93%, greater than about 94%, greater than about 95% or greater than about 96%.

In some embodiments of the present invention, a patient's respiration rate may be measured for a normal rate at rest, i.e., a respiration rate of about 10 to about 20 breaths per minutes. In some embodiments of the present invention, a patient's respiratory rate may be measured for a normal rate, wherein the range of respiratory rates may be selected from the group consisting of greater than about 11 and less than about 20 breaths per minute, greater than about 12 and less than about 20 breaths per minute, greater than about 13 and less than about 20 breaths per minute and greater than about 14 and less than about 20 breaths per minute.

Factors

In some embodiments of the present invention, methods may comprise correlating a normal level of oxygen saturation and a normal rate of respiration with an enhanced risk of opioid-induced respiratory dysfunction if the patient is at an elevated altitude above sea level, wherein the altitude is selected from a range consisting of about 1000 feet above sea level or greater, about 2000 feet above sea level or greater, about 3000 feet above sea level or greater and about 4000 feet above sea level or greater.

In other embodiments, methods may comprise correlating a normal level of oxygen saturation and a normal rate of respiration with an enhanced risk of opioid-induced respiratory dysfunction if the patient's oxygen saturation level is normal but no greater than about 95%, about 94%, about 93%, about 92%, about 91%, or about 90%.

In some embodiments, methods may comprise correlating a normal level of oxygen saturation and a normal rate of respiration with an enhanced risk of opioid-induced respiratory dysfunction if the patient received prior dosing with an intravenous opioid and is converted to dosing with an oral opioid. In other embodiments, the patient is converted to dosing with an oral opioid from a prior intravenous opioid dosing, wherein the prior intravenous opioid dosing was administered for a duration of at least about 48 hours, at least about 24 hours, at least about 20 hours, at least about 16 hours, at least about 12 hours, at least about 8 hours, at least about 6 hours, at least about 4 hours, at least about 2 hours or at least about 1 hour.

In some embodiments, the oral opioid is administered within about 15 minutes of prior intravenous opioid dosing. In some embodiments, the oral opioid is administered within a time range of prior intravenous opioid dosing selected from the group consisting of about 15 minutes to about 30 minutes, about 30 minutes to about 1 hour, about 1 hour to about 2 hours, about 2 hours to about 4 hours, about 4 hours to about 8 hours, about 8 hours to about 12 hours, about 12 hours to about 16 hours, about 16 hours to about 24 hours, and about 24 hours to about 48 hours. In another embodiment, enhanced risk of opioid-induced respiratory dysfunction may occur if a patient is converted to dosing with an oral opioid from a prior intravenous opioid dosing within about 12 hours of cessation of prior intravenous opioid dosing, within about 8 hours of cessation of prior intravenous opioid dosing, within about 4 hours of cessation of prior intravenous opioid dosing, within about 2 hours of cessation of prior intravenous opioid dosing, within about 1 hour of cessation of prior intravenous opioid dosing or within about 30 minutes of cessation of prior intravenous opioid dosing.

In certain embodiments, the prior intravenous opioid dosing comprises one ore more opioids selected from the group consisting of codeine, thebaine, hydromorphone, hydrocodone, oxycodone, oxymorphone, diacetylmorphine (heroin), nicomorphine, dipropanoylmorphine, benzylmorphine, ethylmorphine, buprenorphine and morphine glucuronides (including the 3- and 6-glucuronide), alfentanil, fentanyl, remifentanil, sufentanil, trefentanil, pethidine, methadone, tramadol, dextropropoxyphene, a pharmaceutically acceptable salt thereof, and a combination thereof. In particular embodiments, the prior intravenous opioid dosing is morphine, or a pharmaceutically acceptable salt thereof, or oxycodone, or a pharmaceutical salt thereof.

In certain embodiments, the prior intravenous opioid dosing comprises a combination of at least two opioids. In certain embodiments, the prior intravenous opioid dosing is a combination of two opioids. In certain embodiments, the prior intravenous opioid dosing is a combination comprising morphine, or a pharmaceutically acceptable salt thereof, and oxycodone, or a pharmaceutically acceptable salt thereof. In some embodiments, the prior intravenous opioid dosing comprises morphine, or a pharmaceutically acceptable salt thereof, and oxycodone, or a pharmaceutically acceptable salt thereof in a ratio of about 3:1 to 1:3 (weight:weight). In certain embodiments, the prior intravenous opioid dosing comprises morphine, or a pharmaceutically acceptable salt thereof, and oxycodone, or a pharmaceutically acceptable salt thereof in a ratio of about 3:2 (weight:weight).

In some embodiments, “correlating the normal level of oxygen saturation and the normal rate of respiration with enhanced risk of opioid-induced respiratory dysfunction” refers to associating the normal level of oxygen saturation and the normal rate of respiration with enhanced risk of opioid-induced respiratory dysfunction; or refers to recognizing that the patient having the normal level of oxygen saturation and the normal rate of respiration is at enhanced risk of opioid-induced respiratory dysfunction , or identifying that the patient having the normal level of oxygen saturation and the normal rate of respiration is at enhanced risk of opioid-induced respiratory dysfunction.

In some embodiments of the present invention, the assay for determining the level of oxygen saturation in a patient comprises performing pulse oximetry or an arterial blood gas test.

In certain embodiments, the assay for determining the level of oxygen saturation in a patient comprises performing an arterial blood gas test. In certain embodiments, the assay for determining the level of oxygen saturation in a patient comprises performing pulse oximetry.

Formulations

In certain embodiments of the present invention, the formulation comprises at least one opioid. In some embodiments, the formulation comprises a combination of at least two opioids. In some embodiments, the formulation comprises a combination of two opioids. In certain embodiments, the formulation comprises morphine, or a pharmaceutically acceptable salt thereof, and oxycodone, or a pharmaceutically acceptable salt thereof. In particular embodiments, the formulation comprises morphine, or a pharmaceutically acceptable salt thereof, and oxycodone, or a pharmaceutically acceptable salt thereof in a ratio of about 3:1 to about 1:3 (weight:weight). In certain embodiments, the formulation comprises morphine, or a pharmaceutically acceptable salt thereof, and oxycodone, or a pharmaceutically acceptable salt thereof in a ratio of about 3:2 (weight:weight). In another embodiment, the formulation comprises a combination of morphine sulfate and oxycodone hydrochloride in a ratio of 3:2 (weight:weight). In alternative embodiments, a formulation comprising morphine, or a pharmaceutically acceptable salt thereof, and oxycodone, or a pharmaceutically acceptable salt thereof, are co-administered at about the same time.

In the present invention, the formulations may be administered orally or parenterally. In some embodiment, the parenteral administration is intravenous administration.

In other embodiments, the formulation administered orally may be in an immediate release, sustained release or controlled release dosage form.

In certain embodiments, the pharmaceutically acceptable salt may be a hydrochloride, hydrobromide, hydroiodide, sulfate, bisulfate, nitrate, citrate, tartrate, bitartrate, phosphate, malate, maleate, napsylate, fumarate, succinate, acetate, terephthalate, pamoate or pectinate.

In particular embodiments, the formulation comprises morphine sulfate and oxycodone hydrochloride.

In some embodiments, the formulation may be in an immediate release dosage form, sustained release dosage form, or controlled release dosage form. In particular embodiments, the formulation may be in an immediate release dosage form.

In some embodiments, the IV formulation comprises morphine, codeine, thebaine, hydromorphone, hydrocodone, oxycodone, oxymorphone, diacetylmorphine (heroin), nicomorphine, dipropanoylmorphine, benzylmorphine, ethylmorphine, buprenorphine and morphine glucuronides (including the 3- and 6-glucuronide), alfentanil, fentanyl, remifentanil, sufentanil, trefentanil, pethidine, methadone, tramadol, dextropropoxyphene, a pharmaceutically acceptable salt thereof, or a combination thereof.

In certain embodiments, the IV formulation comprises morphine or oxycodone or a pharmaceutically acceptable salt thereof. In another embodiment, the IV formulation comprises a combination of morphine sulfate and oxycodone hydrochloride in a ratio of about 1:1 (weight:weight). In some embodiments, the formulation comprising at least one opioid has a morphine equivalent dose (MED) of at least about 12 mg, 15 mg , 20 mg, 24 mg, 30 mg, 36 mg, 50 mg, 75 mg, 100 mg or 150 mg.

A suitable combination product of morphine and oxycodone, or pharmaceutically acceptable salts thereof, is disclosed in co-pending U.S. patent application Ser. Nos. 11/544,187, 12/469,438, and 12/567,209.

The formulation for oral administration may be administered in immediate release dosage forms. Immediate release dosage forms such as solid or liquid dosage forms include, by way of example and not limitation, tablets, troches, capsules, dispersions, suspensions, solutions, syrups, and the like. Formulations may be presented as discrete units such as capsules, sachets or tablets, each containing a predetermined amount of each of the opioid, e.g., morphine and oxycodone, or pharmaceutically acceptable salts thereof, as a powder or granules or as a solution or a suspension in an aqueous liquid, a non-aqueous liquid, an oil-in-water liquid emulsion or a water-in-oil liquid emulsion. Such formulations may be prepared by any of the methods of pharmacy but all methods include the step of bringing together each of the opioids with a pharmaceutically acceptable carrier. In general, formulations may be prepared by uniformly and intimately admixing the opioid, e.g., morphine and oxycodone, or pharmaceutically acceptable salts thereof, with liquid carriers or finely divided solid carriers or both, and then, if necessary, shaping the product into the desired presentation. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents together with pharmaceutically active substances is well known in the art. These carriers include, by way of example and not limitation, sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Supplementary active compounds can also be incorporated into the formulations.

Oral formulations generally may include an inert diluent or an edible carrier. Suitable oral formulations may be, e.g., enclosed in gelatin capsules or compressed into tablets, troches, or capsules. For the purpose of oral therapeutic administration, the active compound may be incorporated with excipients and used in the form of tablets, troches, or capsules. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

It is especially advantageous to formulate oral formulations in a unit dosage form for ease of administration and uniformity of dosage. The term “unit dosage form” as used herein refers to physically discrete units suited as unitary dosages for the patient to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The formulations for oral administration may also be administered in controlled release dosage forms. For example, controlled release dosage forms as described hereinafter may be administered every 12- or 24-hours comprising, respectively, about 3 or 6 times the amount of the immediate-release dosage form. In this regard, it is well known that the change from immediate-release dosage forms to controlled-release dosage forms of an opioid, e.g., morphine and oxycodone, or pharmaceutically acceptable salts thereof, may be a simple milligram to milligram conversion that results in the same total “around-the-clock” dose of the morphine and oxycodone, or pharmaceutically acceptable salts thereof. See Cherry and Portenoy, “Practical Issues in the Management of Cancer Pain,” in Textbook of Cancer Pain, Third Edition, Eds. Wall and Meizack, Churchill Livingstone, 1994, 1453.

Controlled-release of the opioid, e.g., morphine and oxycodone, or pharmaceutically acceptable salts thereof, may be affected by incorporating the opioid, e.g., morphine and oxycodone, or pharmaceutically acceptable salts thereof, into, by way of example and not limitation, hydrophobic polymers, including acrylic resins, waxes, higher aliphatic alcohols, polylactic and polyglycolic acids and certain cellulose derivatives, such as hydroxypropylmethyl cellulose. In addition, the controlled release may be affected by using other polymer matrices, liposomes and/or microspheres. The controlled release formulation of opioid, e.g., morphine and oxycodone, or pharmaceutically acceptable salts thereof, may be released at a slower rate and over a longer period of time. For example, in some embodiments, the controlled release formulation of an opioid, e.g., morphine and oxycodone, or pharmaceutically acceptable salts thereof, may release effective amounts of an opioid, e.g., mixture of morphine and oxycodone, or pharmaceutically acceptable salts thereof, over 12 hours. In other embodiments, the controlled release formulation may release effective amounts of an opioid, e.g., morphine and oxycodone, or pharmaceutically acceptable salts thereof, over 4 hours or over 8 hours. In still other embodiments, the controlled release formulation may release effective amounts of an opioid, e.g., morphine and oxycodone, or pharmaceutically acceptable salts thereof, over 15, 18, 24 or 30 hours.

In some embodiments, the controlled release formulation is in accordance to controlled release formulations as described in U.S. application Ser. No. 13/442,849, which is incorporated herein by reference.

An aspect of the invention is a formulation comprising at least one opioid for use in treating pain in a patient at enhanced risk of opioid-induced respiratory dysfunction, wherein the patient has a normal level of oxygen saturation of about 90% or greater, and a normal rate of respiration at rest which is greater than about 10 and less than about 20 breaths per minute, and either: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid; and wherein the formulation comprising as least one opioid is for use in combination with supplemental oxygen if the blood oxygen saturation level is less than about 90% following opioid administration.

Another aspect of the invention is a formulation comprising at least one opioid for use in combination with supplemental oxygen for treating pain in a patient at enhanced risk of opioid-induced respiratory dysfunction, wherein prior to administration of the opioid the patient has a normal level of oxygen saturation of about 90% or greater, and a normal rate of respiration at rest which is greater than about 10 and less than about 20 breaths per minute, and either: (a) the patient is at an altitude of about 1000 feet above sea level or greater; or (b) the patient's oxygen saturation level is normal but no greater than about 95%; or (c) the patient received prior dosing with intravenous opioid and is converted to dosing with an oral opioid; and wherein following administration of the opioid the blood oxygen saturation level drops to less than about 90%.

### EXAMPLES

### Example 1

Three double blind, randomized clinical studies were conducted comparing the effects of MoxDuo®, a fixed dose combination of morphine sulfate and oxycodone hydrochloride in a ratio of about 3:2 (weight:weight), morphine sulfate alone, and oxycodone hydrochloride alone during treatment of moderate to severe postoperative pain following bunionectomy surgery. The three studies are summarized in Table 1.

TABLE 1
Study Designs.

| | Study 007 | Study 008 | Study 021 |
|---|---|---|---|
| Study | Double-blind, | Double-blind, | Double-blind, |
| Design | randomized, | randomized, | randomized, |
| | placebo-controlled | repeat-dose | fixed dose, |
| | | active- | active-controlled |
| | | controlled | |
| Randomized | 256 | 522 | 197 |
| Patients | | | |
| Treatment | MoxDuo: 3/2, 6/4, | MoxDuo: 12/8; | MoxDuo: 6/4, 12/8; |
| Groups (mg) | 12/8, 18/12; | morphine: 12; | morphine: 6, 12; |
| | Placebo | oxycodone: 8 | oxycodone: 4, 8 |
| Morphine | 12, 24, 36 | 12, 24 | 12, 24 |
| equivalent | | | |
| doses (mg) | | | |

In total, 975 patients were involved in these studies, which included 60 patients administered a placebo, 433 patients administered MoxDuo, and 482 patients administered either morphine alone or oxycodone alone. In the study, 71.5% of MoxDuo patients received an MED that was at least twice that of morphine or oxycodone groups (Table 2). Thus, the presence of higher MED of MoxDuo provides a conservative bias (against MoxDuo) in the analyses.

TABLE 2
Mean Morphine Equivalent Dose Exposure of Treatment Groups.

| | MoxDuo | | | Morphine | | Oxycodone | |
|---|---|---|---|---|---|---|---|
| Morphine Equivalent | 12 | 24 | ≧30 | 6-12 | 24 | 6-12 | 24 |
| Dose (mg) | | | | | | | |
| Number of Patients | 80 | 304 | 49 | 242 | 0 | 240 | 0 |
| Exposed (n) | | | | | | | |

Oxygen saturation was measured continuously during baseline and the 48 hour treatment periods using pulse oximetry. The total number and percentage of patients with oxygen desaturation (SpO₂<90%) were recorded at: 0, 15, 30 and 45 minutes, and 1, 2, 3, 4, 5, 6 and 8 hours after study drug administration; at the time of administration of subsequent doses of study medication; at the time of administration of supplemental analgesic medication; 1 hour after administration of study medication; and whenever a desaturation occurred during the dosing period.

Respiration rate (<10 breaths/min, 10-12 breaths/min, >12 breaths/min) was summarized at the same time points as the SpO₂ recordings. Cumulative MED was determined at the time of the first episode of SpO₂<90%. All desaturations and use of supplemental oxygenation, use of narcotic antagonists against respiratory impairment, and respiratory related serious adverse events (SAEs) were recorded.

In subjects with oxygen desaturation the following data was recorded: cumulative MED at first desaturation; mean desaturation values; number of desaturation episodes; and total duration of desaturation episodes.

Some patients in the three clinical studies were enrolled who resided near study sites that are located at an altitude of about or over 4000 feet above sea level. These patients all exhibited near normal baseline respiratory function (SpO₂≧90%, respiration rate at rest of about 10 to about 20 breaths/min) and had no other signs of clinically significantly impaired respiratory dysfunction.

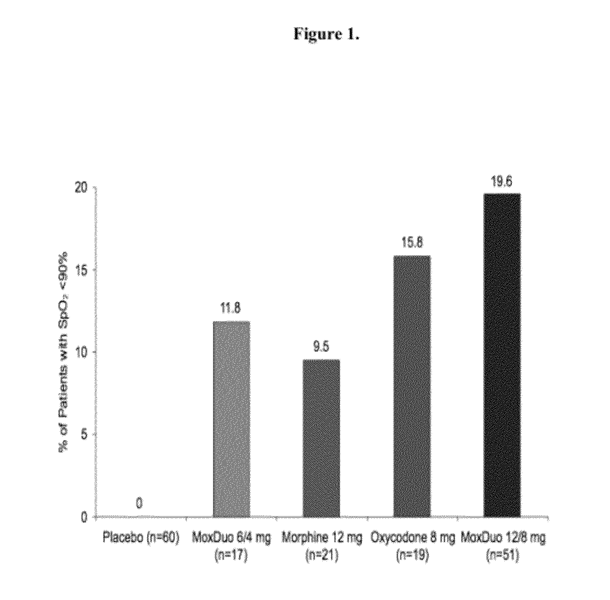

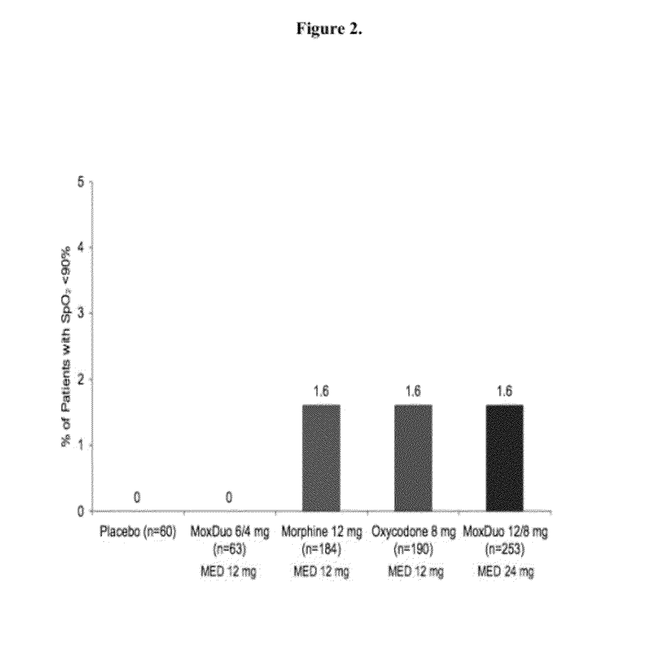

Compared to patients enrolled at sites at near sea-level, the incidence of desaturation was greater for each opioid treatment, despite that the dosages were the same (compare FIG. 1 and FIG. 2). For example, the incidence of desaturation induced by administration of 12 mg MED of MoxDuo was 11.8% at the higher elevation sites, compared to 0.0% at the lower elevation sites. Similarly, when 20-24 mg MED of MoxDuo was administered, incidence of desaturation was over ten-fold greater at the high elevation sites as compared to the lower elevation sites (19.6% vs. 1.6%). Moreover, incidence of desaturation induced by morphine or oxycodone administered at the high elevation site was nearly six-fold and ten-fold greater, respectively, as compared to incidence of desaturation induced by administration at the lower sites.

These results demonstrate that, despite having normal (baseline) respiratory function, patients residing at high elevations have a substantially greater respiratory dysfunction than patients at lower elevations. These results are surprising, as patients were acclimated to the high altitudes due to residence at the site locations and their normal (baseline) respiratory function negates any expectation that altitude would have an effect on the risk of opioid-induced respiratory dysfunction.

### Example 2

A clinical trial was conducted to study the administration of oral doses of MoxDuo to patients who received intravenous dosing with morphine immediately following surgery before patients were able to orally ingest medication. Following surgery, baseline oxygen saturation levels and respiration rate were measured to confirm that the patients' baseline respiratory function was normal. Once patients were able to orally ingest medication, IV dosing of opioid was discontinued and orally administered MoxDuo at doses determined by an algorithm described in U.S. Pat. No. 7,923,453, which is herein incorporated by reference.

The total number and percentage of subjects with oxygen desaturation (SpO₂<90%) was recorded and correlated with baseline oxygen saturation levels. Table 3 shows that a patient with a near normal baseline oxygen saturation level at or below 95% has an appreciably greater likelihood of experiencing opioid-induced oxygen desaturation than a patient with normal baseline oxygen saturation level that is above 95%

TABLE 3
Ratio of patients experiencing no-desaturation to desaturation
as a function of baseline oxygen saturation.

| | | Ratio of patients |
|---|---|---|
| | | experiencing no |
| | | desaturation/patients |
| Baseline SpO₂ | Total number of patients | experiencing desaturation |
| 94-95% | 27 | 1.45 |
| 95% | 26 | 1.60 |
| 96% | 29 | 6.25 |
| 97% | 36 | 5.00 |
| 98% | 26 | 5.50 |
| 99% | 23 | 4.75 |

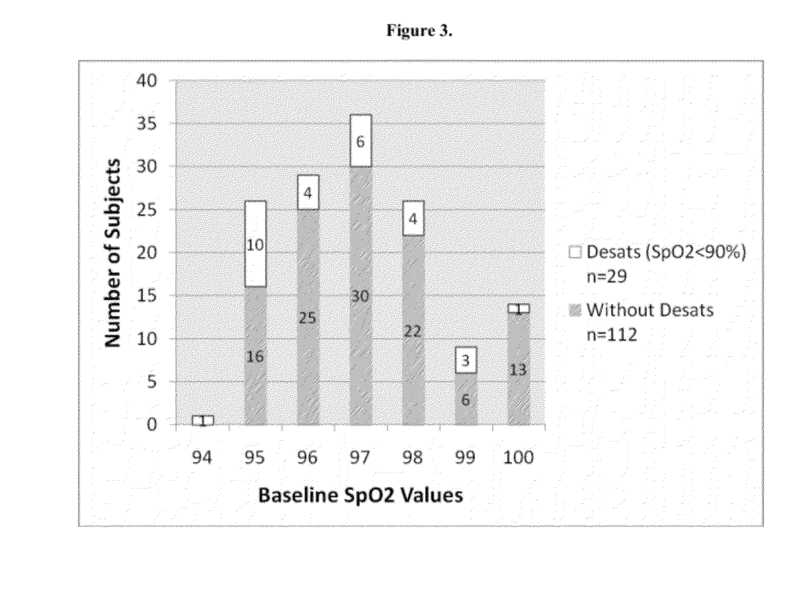

Patients with a baseline oxygen saturation of greater than or equal to 94%-95%, but less than 96% (SpO₂>94%, but <96%) were compared with patients having oxygen saturation of greater than or equal to 96% (SpO₂>96%) (FIG. 3). The results suggest that patients with a normal baseline oxygen saturation level of about 95% are nearly 3-4 times as likely to experience oxygen desaturation upon administration of oral MoxDuo than patients with a normal baseline oxygen saturation level of 96% or above (odds ratio of 1.65) vs. 4.75-6.25).

### Example 3

The study described Example 2 showed that, in patients who were age ≧60 and received an oral opioid (mean of 12 mg MED), over 20% experienced an oxygen desaturation event that occurred at a mean average of 6.5 hours after the onset of dosing (Table 4). Comparing these results to other patients of a separate study who were age ≧60 and did not receive prior intravenous dosing of an opioid but who received on oral opioid of 12 mg MED, the former group had an elevated incidence of desaturation. Moreover, the mean onset time after the first dose of opioid medication at which a desaturation occurred was shorter in patients who received prior intravenous opioid dosing (Table 4). In particular, the likelihood of oxygen desaturation was nearly 100% greater for patients who were previously administered intravenous opioid doses compared to similar patients who had not received prior intravenous opioid doses.

TABLE 4
Desaturation (SpO₂ < 90%) risk and onset: following initiation
of oral opioid dosing, with or without prior IV PCA Morphine.

| | No prior | Prior intravenous |
|---|---|---|
| | intravenous dosing | dosing |
| Patients (no.) | 47 | 142 |
| Mean age (yrs) | 63 | 66 |
| No. of patients who experienced | 5 | 29 |
| oxygen desaturation | | |
| % of patients who experienced | 10.6% | 20.4% |
| oxygen desaturation | | |
| Mean time to oxygen | 11.4 | 6.5 |
| desaturation* (hrs) | | |
| Median time to oxygen | 8.8 | 3.5 |
| desaturation* (hrs) | | |

*determined from patients who experienced oxygen saturation

### Example 4

The study described in Example 1 showed that patients who received MoxDuo generally required a higher cumulative dose (72 mg) before experiencing an oxygen desaturation event (SpO₂<90%) when compared to those subjects who received either morphine or oxycodone alone (24 mg). Additionally, of patients who experienced oxygen desaturation, the median number of episodes of SpO₂<90% for MoxDuo was 1.5, compared with 2.0 for morphine or oxycodone alone.

At non-high altitude sites, the rate of incidence of oxygen desaturation for 24 mg MED of MoxDuo (1.6%) is identical to the rate of incidence of oxygen desaturation of 12 mg MED for morphine (1.6%) and oxycodone (1.6%) (FIG. 2). Moreover, 0% of patients in the group receiving an MED of 12 mg of MoxDuo had an oxygen desaturation event, compared to 12 mg MED morphine (1.6%) and oxycodone (1.6%).

Having thus described in detail embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for detecting enhanced risk of opioid-induced respiratory dysfunction in a patient to be treated for pain, comprising:
(1) assaying blood of the patient for a normal level of oxygen saturation, wherein the normal level of oxygen saturation is about 90% or greater;
(2) measuring the patient's respiration for a normal rate, wherein the normal rate of respiration at rest is greater than about 10 and less than about 20 breaths per minute; and
(3) correlating the normal level of oxygen saturation and the normal rate of respiration with enhanced risk of opioid-induced respiratory dysfunction if:
(a) the patient is at an altitude of about 1000 feet above sea level or greater; or
(b) the patient's oxygen saturation level is normal but no greater than about 95%; or
(c) the patient received prior dosing with intravenous opioid and is converted to dosing with an oral opioid.

2. The method of claim 1, wherein if the patient to be treated for pain is at an altitude of about 1000 feet above sea level or greater, then the patient is to be administered an oral or parenteral opioid formulation comprising at least one opioid.

3. The method of claim 1, wherein if the patient to be treated for pain has an oxygen saturation level that is normal but no greater than about 95%, then the patient is to be administered an oral or parenteral opioid formulation comprising at least one opioid.

4. The method of claim 1, wherein the assay for the normal level of oxygen saturation comprises performing pulse oximetry or an arterial blood gas test.

5. The method of claim 1, wherein the patient to be treated for pain is at enhanced risk of opioid-induced respiratory dysfunction if the patient is at an altitude of 2000 feet above sea level or greater.

6. The method of claim 1, wherein the patient to be treated for pain is at enhanced risk of opioid-induced respiratory dysfunction if the patient is at an altitude of 3000 feet above sea level or greater.

7. The method of claim 1, wherein the patient to be treated for pain is at enhanced risk of opioid-induced respiratory dysfunction if the patient is at an altitude of 4000 feet above sea level or greater.

8. The method of claim 1, wherein the patient to be treated for pain is to be administered a formulation comprising at least one opioid.

9. The method of claim 8, wherein the formulation comprises at least two opioids.

10. The method of claim 9, wherein the formulation comprises morphine, or a salt thereof, and oxycodone, or a salt thereof.

11. The method of claim 10, wherein the morphine, or a salt thereof, and oxycodone, or a salt thereof, are in ratio of 3:2 by weight.

12. The method of claim 2, wherein the parenteral administration is intravenous.

13. The method of claim 3, wherein the parenteral administration is intravenous.

14. A method for method for administering at least one opioid to a patient comprising:
(1) assaying blood of the patient for a normal level of oxygen saturation, wherein the normal level of oxygen saturation is about 90% or greater;
(2) measuring the patient's respiration for a normal rate, wherein the normal rate of respiration at rest is greater than about 10 and less than about 20 breaths per minute; and
(3) correlating the normal level of oxygen saturation and the normal rate of respiration with enhanced risk of opioid-induced respiratory dysfunction if:
(a) the patient is at an altitude of about 1000 feet above sea level or greater; or
(b) the patient's oxygen saturation level is normal but no greater than about 95%; or
(c) the patient received prior dosing with intravenous opioid and is converted to dosing with an oral opioid;
(4) administering to the patient a formulation comprising at least one opioid; and
(5) monitoring the patient's oxygen saturation level if the patient is determined to be at enhanced risk of opioid-induced respiratory dysfunction, and administering supplemental oxygen if the oxygen saturation level is less than about 90%.

15. The method of claim 14, wherein if the patient to be treated for pain is at an altitude of about 1000 feet above sea level or greater, then the patient is administered an oral or parenteral opioid formulation comprising at least one opioid.

16. The method of claim 14, wherein if the patient to be treated for pain has an oxygen saturation level that is normal but no greater than about 95%, then the patient is administered an oral or parenteral opioid formulation comprising at least one opioid.

17. The method of claim 14, wherein the assay for a normal level of oxygen saturation comprises performing pulse oximetry or an arterial blood gas test.

18. The method of claim 14, wherein the patient is at enhanced risk of opioid-induced respiratory dysfunction if the patient is at an altitude of 2000 feet above sea level or greater.

19. The method of claim 14, wherein the patient is at enhanced risk of opioid-induced respiratory dysfunction if the patient is at an altitude of 3000 feet above sea level or greater.

20. The method of claim 14, wherein the patient is at enhanced risk of opioid-induced respiratory dysfunction if the patient is at an altitude of 4000 feet above sea level or greater.

21. The method of claim 14, wherein the formulation comprises at least two opioids.

22. The method of claim 21, wherein the formulation comprises morphine, or a salt thereof, and oxycodone, or a salt thereof.

23. The method of claim 22, wherein the morphine, or a salt thereof, and oxycodone, or a salt thereof, are in ratio of about 3:1 to about 1:3, by weight.

24. The method of claim 15, wherein the parenteral administration is intravenous.

25. The method of claim 16, wherein the parenteral administration is intravenous.

26. A method of treating pain in a patient with an enhanced risk of opioid-induced respiratory dysfunction, which comprises:
(1) identifying a patient with an enhanced risk of opioid-induced respiratory dysfunction by:
(a) assaying blood of the patient for a normal level of oxygen saturation, wherein the normal level of oxygen saturation is about 90% or greater;
(b) measuring the patient's respiration for a normal rate, wherein the normal rate of respiration at rest is greater than about 10 and less than about breaths per minute; and
(c) correlating the normal level of oxygen saturation and the normal rate of respiration with enhanced risk of opioid-induced respiratory dysfunction if:
(i) the patient is at an altitude of about 1000 feet above sea level or greater; or
(ii) the patient's oxygen saturation level is normal but no greater than about 95%; or
(iii) the patient received prior dosing with intravenous opioid and is converted to dosing with an oral opioid;
(2) administering a formulation of two or more opioids concurrently to the patient if the patient is determined to be at enhanced risk of opioid-induced respiratory dysfunction.

27. The method of claim 26, wherein if the patient with an enhanced risk of opioid-induced respiratory dysfunction is at an altitude of about 1000 feet above sea level or greater, then the patient is administered an oral or parenteral formulation.

28. The method of claim 26, wherein if the patient with an enhanced risk of opioid-induced respiratory dysfunction has an oxygen saturation level that is normal but no greater than about 95%, then the patient is administered an oral or parenteral formulation.

29. The method of claim 26, wherein the formulation of two or more opioids comprises morphine, or a salt thereof, and oxycodone, or a salt thereof.

30. The method of claim 29, wherein the morphine, or a salt thereof, and oxycodone, or a salt thereof, are in ratio of about 3:1 to about 1:3, by weight.

31. The method of claim 27, wherein the parenteral administration is intravenous.

32. The method of claim 28, wherein the parenteral administration is intravenous.

33. The method of claim 26, wherein the patient is at an altitude of 4000 feet above sea level or greater.
